(19) United States
(12) Patent Application Publication
(10) Pub. No.: US 2014/0114616 A1
(43) Pub. Date: Apr. 24, 2014

(54) SYSTEM AND METHOD FOR PARAMETERIZING SIGNALS WITH FINITE-RATES-OF-INNOVATION

(71) Applicant: QUALCOMM Incorporated, San Diego, CA (US)

(72) Inventors: Chong Uk LEE, Cupertino, CA (US); John Hyunchul HONG, San Clemente, CA (US)

(73) Assignee: QUALCOMM Incorporated, San Diego, CA (US)

(21) Appl. No.: 14/058,065

(22) Filed: Oct. 18, 2013

Related U.S. Application Data
(60) Provisional application No. 61/785,679, filed on Mar. 14, 2013.
(60) Provisional application No. 61/717,491, filed on Oct. 23, 2012.

(51) Int. Cl. G06F 15/00; A61B 5/04

(52) U.S. Cl. CPC G06F 15/00; A61B 5/04012
USPC 702/190; 702/189; 600/509

(57) ABSTRACT

Systems and methods are described herein for defining and parameterizing signals or system responses with finite rate of innovation (FRI) signal processing. A delta-sigma modulator is used at a low sampling rate to digitize an analog signal for FRI processing. This allows for reduced or eliminated analog pre-filtering while still utilizing low sample rates for an overall reduction in circuit size and power dissipation over conventional FRI signal acquisition techniques.

### CROSS REFERENCE TO RELATED APPLICATIONS

This application claims priority under 35 U.S.C. Section 119(e) to U.S. Provisional Application 61/717,491, filed on Oct. 23, 2012, and U.S. Provisional Application 61/785,679, filed on Mar. 14, 2013.

### TECHNICAL FIELD

This disclosure relates to methods of acquiring and processing signals for finite rate of innovation parameterization.

### DESCRIPTION OF THE RELATED TECHNOLOGY

Signal parameterization is widely used in signal processing, storage, transmission, and analysis. Perhaps the most common is the use of Nyquist rate sampling, where a continuous time domain signal is represented by a set of sampled signal values at discrete times. As long as the original continuous signal is band limited to at most half the sampling rate, the set of samples can be used to reconstruct the complete signal by using, for example, a sine interpolation algorithm. In this common example, the signal is represented by a set of discrete parameters, the sample values, which can be stored, transmitted, and used at any time to reconstruct the original signal.

More recently, some signals having a large or even theoretically infinite bandwidth have been parameterized in other ways. Although these signals may contain frequency components that are arbitrarily large, they are characterized by a “rate-of-innovation” per unit time so that the signal can be parameterized with a finite set of values from which the original signal can be reconstructed. The problem to be solved then is how to derive a suitable set of parameter values from the original signal and how to reverse the process to reconstruct the complete signal using only the derived parameters. Finite Rate of Innovation (FRI) based signal analysis is one such method of analyzing signals, and is described in the following references: [1] M. Vetterli, P. Marziliano, T. Blu, “Sampling Signals with Finite Rate of Innovation”, IEEE Transactions on Signal Processing, vol. 50, no. 6, pp. 1417-1428, June 2002; [2] T. Blu, P. L. Dragotti, M. Vetterli, P. Marziliano, and L Coulot, “Sparse Sampling of Signal Innovations: Theory, Algorithms, and Performance Bounds”, IEEE Signal Processing Magazine, vol. 25, no. 2, pp. 31-40, March 2008; [3] Y. Hao, P. Marziliano, M. Vetterli, T. Blu, “Compression of ECG as a Signal with Finite Rate of Innovation”, Proc. of the 2005 IEEE Engineering in Medicine and Biology 27^{th} Annual Conference, Shanghai, China, Sep. 1-4, 2005, pp. 7564-7567; [4] Marziliano, M. Vetterli and T. Blu, “Sampling and Exact Reconstruction of Bandlimited Signals With Additive Shot Noise,” IEEE Transactions on Information Theory, vol. 52, No. 5, pp. 2230-2233, May 2006, and [5] U.S. Patent Publication 2011/0225218 to Eldar et al. entitled Low-Rate Sampling of Pulse Streams. Each of these references is incorporated by reference herein in its entirety.

As one example, a signal may contain a sequence of narrow pulses, where the information content in the signal is encoded in the location and amplitude of the pulses in the signal. If the pulse widths are narrow, conventional Nyquist rate sampling for storage, analysis, and reconstruction of such a signal would require high speed, high resolution sampling and storage of a large number of samples. However, because the signal may be characterized with only a few relevant parameters, such as a location and amplitude for each pulse, such a signal has a “rate of innovation” that is small. It has been shown (see reference 1 above, for example) that for such a signal, the relevant parameters of location and amplitude for each pulse in the signal can be derived using only low frequency components of the original signal. The frequency components required to derive the relevant parameters depends on the rate of innovation of the input signal rather than on the bandwidth of the input signal. As used herein, “FRI processing” means a process of using a subset of the frequency components of a signal, usually low frequency components, to derive parameters from which the original signal, including components of higher frequency than those used to derive the parameters, can be reconstructed. The derived parameters are referred to herein as “FRI parameters.” Pulse locations and amplitudes are examples of FRI parameters. This may be contrasted with what may be referred to as “Nyquist processing” and “Nyquist parameters,” which involve time or frequency domain data points having a density that satisfies the Nyquist sampling criteria for the bandwidth of the original signal.

FIGS. 1A and 1B are simple block diagrams of conventional circuits that may be used to sample and process an input signal for FRI processing. In FIG. 1A, an input waveform 12 is sampled by a sampler 14, which may be an A/D converter. The input waveform 12 may be characterized by a bandlimit of fₘ, which is the highest frequency present in the input signal 12. Digital samples output from the sampler 14 are routed to a processing circuit 16 that is configured to implement FRI processing. As described above, the processing circuit 16 may extract low frequency components of the input signal 12 by processing the acquired samples, and use these low frequency components to further derive FRI parameters for storage and/or subsequent reconstruction of the original signal 12. As also described above, the amount of data in the FRI parameters may be much less than the amount of data in the set of acquired samples, producing signal compression for efficient storage, transmission and signal reconstruction. In the implementation of FIG. 1A, the sampler must have a sampling rate of at least 2fₘ, since even though the FRI processing uses only low frequency components of the signal 12, a sampling rate of at least the Nyquist rate must be used to avoid signal distortion. Because FRI processing is typically applied to signals 12 with a large fₘ, sometimes theoretically infinite, the circuit of FIG. 1A may be impractical to physically implement.

For this reason, and as shown in FIG. 1B, proposed implementations of sample acquisition for FRI processing typically involve an analog filter 22 that applies a “sampling kernel” to the input signal 12. The sampling kernel may be a simple “brick wall” low-pass filter, although other sampling kernels have been proposed (for example in references [1] and [5] above). The bandwidth of this filter 22 may be based on the rate of innovation of the signal 12, which determines the frequency components required to generate the FRI parameters. Because the output of the filter 22 is bandlimited based on the rate of innovation, an A/D converter 24 connected to the filter output can use a sampling rate as low as 2f_{FRI}, where f_{FRI} is the highest frequency component necessary to produce the FRI parameters, which may be the cutoff frequency of the filter 22, and which is typically much less than the bandlimit fₘ of the original signal 12. This ability to use a sampling rate lower than the Nyquist rate for the original signal 12 is considered a major benefit of FRI processing.

### SUMMARY

The systems, methods, and devices of the invention each have several aspects, no single one of which is solely responsible for its desirable attributes. Without limiting the scope of this invention as expressed by the claims which follow, some features will now be discussed briefly. After considering this discussion, and particularly after reading the section entitled “Detailed Description” one will understand how the features of this invention provide advantages that include lower power signal acquisition circuitry for use in FRI processing.

In one implementation, a method of sparse sampling an analog signal includes sampling and quantizing the analog signal with a delta-sigma modulator, filtering the sampled and quantized analog signal, and deriving FRI parameters from the filtered, sampled, and quantized analog filter.

In another implementation, an apparatus for parameterizing an analog signal includes a delta-sigma modulator having an input coupled to receive the analog signal and processing circuitry coupled to an output of the delta-sigma modulator and configured to filter an output of the delta-sigma modulator and to derive FRI parameters from the output of the delta-sigma modulator.

In another implementation, a method for sparse sampling an analog signal includes sampling and quantizing the analog signal with a delta-sigma modulator, determining a set of DFT coefficients from the output of the delta-sigma modulator, and deriving FRI parameters from the set of DFT coefficients

In another implementation, an apparatus for sparse sampling an analog signal includes a delta-sigma modulator having an input coupled to receive the analog signal, and processing circuitry coupled to an output of the delta-sigma modulator, and configured to determine a set of DFT coefficients from the output of the delta-sigma modulator and derive FRI parameters from the set of DFT coefficients.

### BRIEF DESCRIPTION OF THE DRAWINGS

FIG. 1A is a block diagram of a prior art signal acquisition circuit for FRI processing.

FIG. 1B is a block diagram of another prior art signal acquisition circuit for FRI processing.

FIG. 2 is a block diagram of a circuit for signal acquisition for FRI processing that includes a delta-sigma modulator.

FIGS. 3A and 3B are block diagrams of example delta-sigma modulator circuits.

FIG. 4 is another circuit for signal acquisition for FRI processing that includes a delta-sigma modulator.

FIG. 5 is a circuit for signal acquisition for FRI processing that includes a delta-sigma modulator with a circuit for DFT computation of the delta-sigma modulator output.

FIG. 6 is a block diagram of an ambulatory ECG monitor using a delta-sigma modulator with FRI processing.

### DETAILED DESCRIPTION

Various aspects of the novel systems, apparatuses, and methods are described more fully hereinafter with reference to the accompanying drawings. The teachings may be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the invention. For example, an apparatus may be implemented or a method may be practiced using any number of the aspects set forth herein. In addition, the scope of the invention is intended to cover such an apparatus or method which is practiced using other structure, functionality, or structure and functionality in addition to or other than the various aspects of the invention set forth herein. It should be understood that any aspect disclosed herein may be embodied by one or more elements of a claim.

Although particular aspects are described herein, many variations and permutations of these aspects fall within the scope of the disclosure. Although some benefits and advantages of the preferred aspects are mentioned, the scope of the disclosure is not intended to be limited to particular benefits, uses, or objectives. Rather, aspects of the disclosure are intended to be broadly applicable to different systems, some of which are illustrated by way of example in the figures and in the following description of the preferred aspects. The detailed description and drawings are merely illustrative of the disclosure rather than limiting, the scope of the disclosure being defined by the appended claims and equivalents thereof.

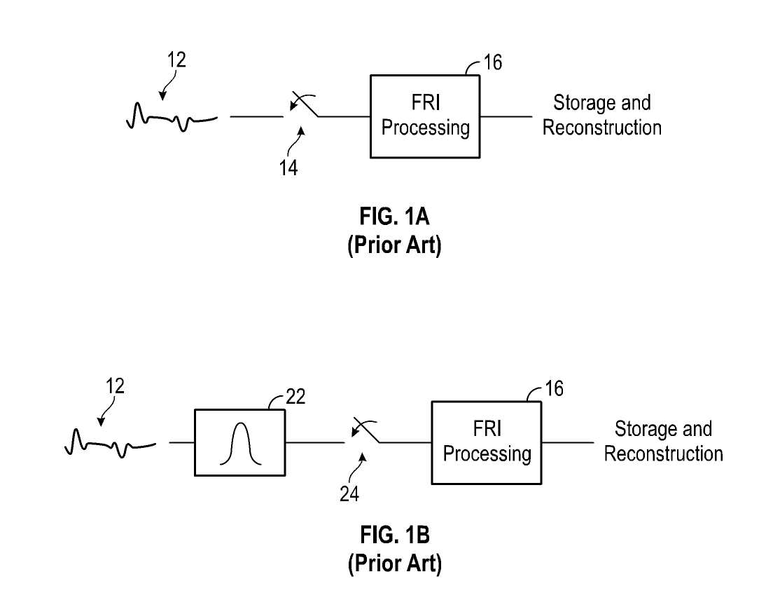

As described above, the circuit of FIG. 1B has been the most commonly proposed implementation of FRI signal acquisition and processing. Because the rates of innovation of many signals are low (even if the bandwidth of interest is large), it is theoretically possible to use low sampling rates for the sampler 24 if the signal is appropriately analog filtered prior to sampling. This has the benefit of reduced complexity and lower power operation for the sampler 24 and reduced amounts of data to process for the processing circuit 16. This is especially valuable, for example, in battery powered on-body monitoring systems where low power operation is of high importance. However, to take full advantage of the low potential sampling rates, the analog filter 22 must have a low cutoff frequency and operate at high dynamic range, which requires large reactive components and/or power dissipating active filter circuitry. Relaxing these requirements on the filter 22 requires use of more complex and power hungry sampler circuitry 24 and produces more data for the processing circuit 16 to handle.

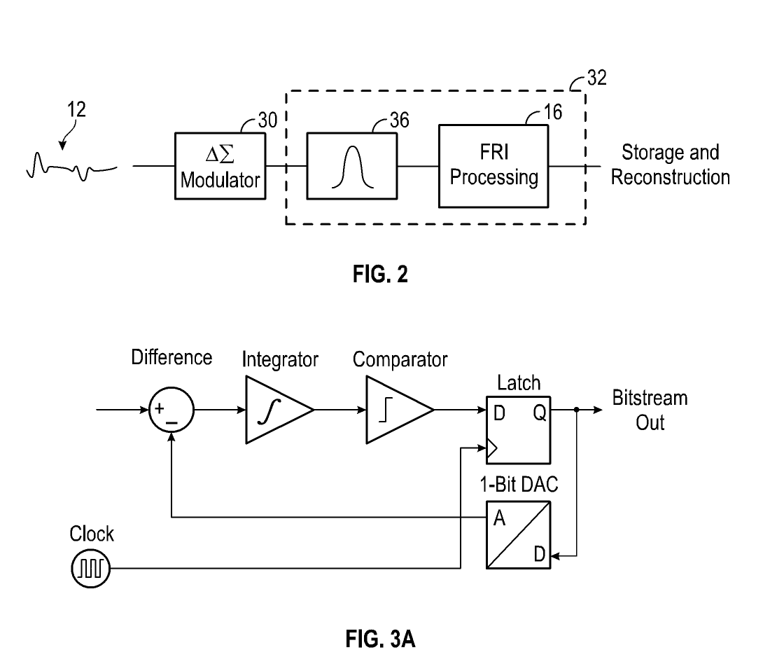

FIG. 2 is a block diagram of a signal acquisition and processing circuit that performs the sample acquisition functions of FIGS. 1A and 1B with much less power dissipation than is generally possible with the circuits of FIGS. 1A and 1B. In FIG. 2, the input signal 12 is sampled directly by a ΔΣ modulator 30 without (or at least with minimal) pre-filtering in the analog domain. In this implementation, a digital processing circuit 32 may apply the sampling kernel 36 by digitally filtering the output of the ΔΣ modulator 30 with the desired function for extracting the frequency components of the signal 12 that are used to derive the FRI parameters. FRI processing can then proceed without modification as described above. This greatly reduces or eliminates the front-end analog filtering used in the system, reducing cost, size, and power dissipation arising from active filter components. ΔΣ modulators are well known signal digitizing circuits, and low pass filtering/decimation is often performed on their output to produce digital data samples representing an input analog signal. However, the inventors have realized that their application to signal acquisition for FRI processing can be especially advantageous when implemented with the appropriate sampling rate.

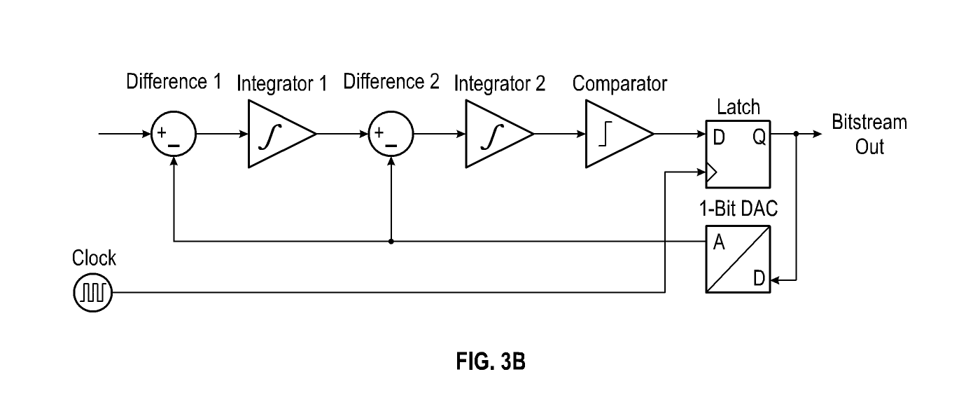

Example ΔΣ modulator circuits are provided in FIGS. 3A and 3B for reference. In general, a ΔΣ modulator is a low resolution (typically two level, or a single bit, but could be more) high sample rate sampler. They include negative output feedback (the Δ part) and at least one integrator (the Σ part) in the signal path, and any A/D converter having these features is considered a “ΔΣ modulator” as used herein. FIG. 3A is an example of a first-order ΔΣ modulator, and FIG. 3B is an example of a second order ΔΣ modulator. Higher order ΔΣ modulators with additional integration stages are also well known in the art.

In a normal implementation of signal processing with a ΔΣ modulator, the sample rate of the ΔΣ modulator is oversampled at a multiple of the Nyquist rate for the highest frequency content fₘ of the input signal. A value referred to as the oversampling ratio (OSR) is often defined as fₛ/2fₘ, where fₛ is the sampling rate of the ΔΣ modulator (e.g. the clock rate of the clock in FIGS. 3A and 3B). As long as the OSR is more than 1, the Nyquist condition is satisfied, and the sampling does not produce signal distortion from aliasing. However, because the ΔΣ modulator has a very low resolution output, usually a single bit, there is a large amount of quantization noise when a ΔΣ modulator is used near the Nyquist rate. An OSR much larger than 1 and low pass filtering after sample acquisition reduce quantization noise to dramatically improve the signal to noise ratio (SNR) in the frequency band of interest, which is fₘ and lower. The ΔΣ modulator design and the OSR used with it may be selected to achieve a particular SNR, which may be at least 70 dB. A SNR of 70-100 dB is suitable for many applications. For second and higher order ΔΣ modulators, an OSR of 16 to 64 can generally achieve this. A SNR of 120 dB or more is also achievable, usually using higher order ΔΣ modulators. Generally, the higher order the ΔΣ modulator, the lower OSR can be used to get a particular SNR output.

The above discussion describes the usual use of ΔΣ modulators, where the highest input signal frequency content fₘ is determined, and a ΔΣ modulator design and OSR are selected based on fₘ to digitize the input signal with a desired SNR. Such a use of a ΔΣ modulator in an FRI processing system may eliminate the need for significant analog pre-filtering. However, the use of a ΔΣ modulator in this way does not fully utilize the characteristics of ΔΣ modulators that can be taken advantage of in an FRI processing system, as fₘ is already very high for these types of signals. In these cases, oversampling with a ΔΣ modulator may be difficult, for the same reasons that implementing the Nyquist rate high resolution sampler illustrated in FIG. 1A may be difficult.

The inventors have realized, however, that as long as the OSR of the ΔΣ modulator 30 in FIG. 2 is at least 1 so that the sampling does not distort the input signal 12, the OSR can be based on the desired SNR and the frequency content needed for the FRI analysis, rather than the desired SNR and the frequency content of the original signal. Conventionally, the sampling frequency fₛ of a ΔΣ modulator is selected to be 2(fₘ)(OSR), where fₘ is the highest frequency content of the input signal and the OSR is selected based on the desired SNR and design of the ΔΣ modulator (e.g. first order, second order, etc.). In the implementation of FIG. 2, the sampling frequency fₛ of the ΔΣ modulator may be selected as 2(f_{FRI})(OSR) where f_{FRI} is the highest frequency content needed for the FRI analysis, and the OSR is selected the conventional way based on the desired SNR and design of the ΔΣ modulator, with the additional requirement that fₛ is at least 2fₘ. As described above, f_{FRI} is generally much lower than fₘ, so that the sampling rate of the ΔΣ modulator 30 of FIG. 2 can be much lower than would conventionally be considered. Although the actual OSR is defined as fₛ/2fₘ, due to low pass filtering in the digital domain to the frequencies needed by the FRI processing, an “effective OSR” can be defined as fₛ/2f_{FRI}. Thus, the ΔΣ modulator can be operated at very low actual OSR, while the effective OSR is still 16, 32, 64, or more to produce the desired SNR in the FRI band of interest. In many implementations, an actual OSR of 1-8 may be used, in some implementations 1-2, in some implementations 1-1.1, all of which are much lower than a conventional use of a ΔΣ modulator, but which can still produce an effective OSR of 32, 64, or more, so that an undistorted, high SNR digital output is produced for input to the FRI processing.

As an example, consider an input signal with fₘ of 3 kHz and an FRI processing algorithm that requires frequency components from 0 to 50 Hz to derive the FRI parameters that can reconstruct the original 3 kHz signal waveform. Conventionally, such a signal could be sampled unfiltered at 6 kHz or more using a Nyquist rate high resolution (e.g. 12-16 bit) sampler as in FIG. 1A. Alternatively, as in FIG. 1B, the signal could be low pass filtered to 50 Hz, then sampled at 100 Hz with a Nyquist rate high resolution sampler. Each of these implementations may produce a 70-100 dB SNR in the 0-50 Hz band of interest that can be used in the FRI processing. With the circuit of FIG. 2, however, a single bit output ΔΣ modulator with a sampling rate of 6.4 kHz with digital low pass filtering may be used to produce a 70-100 dB SNR in the 0-50 Hz band of interest. In this case, the actual OSR of the ΔΣ modulator is only 1.06, but the effective OSR is 64, and this can still produce a 70-100 dB SNR in the 0-50 Hz band of interest. With the sampling scheme of FIG. 2, the power requirements of the circuit can be 2 to 5 times lower than the implementations of FIG. 1A or 1B, and no large reactive filter components are needed as in the circuit of FIG. 1B.

Although not illustrated, some analog pre-filtering can also be used in the system of FIG. 2. However, the requirements on any such filtering are much more relaxed than what would normally be implemented in the conventional system of FIG. 1B. In such a case, the fₛ necessary to produce an effective OSR of 16, 32, 64, or other desired value may be computed based on f_{FRI}. The input signal 12 may then be low pass filtered to have a highest frequency component of equal to or somewhat less than half this fₛ. This makes the actual fₛ near the lower limit of 2fₘ and an actual OSR of near 1, where fₘ is now the highest frequency content of the filtered signal input to the ΔΣ modulator.

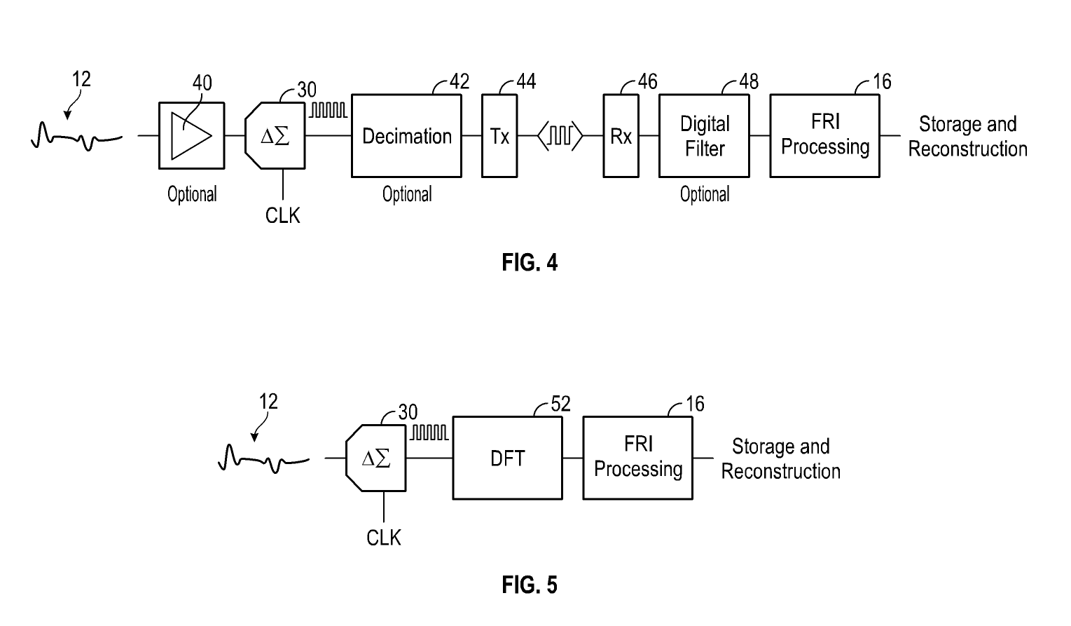

FIG. 4 illustrates an implementation of the circuit of FIG. 2 in a system that includes transmission and reception of the sampled data prior to FRI processing. The signal 12 may optionally be input to an amplifier 40 which may include some analog filtering as described above. The output of the amplifier 40 is sampled at the ΔΣ modulator 30 as described above. Decimation filtering can optionally be applied by digital filter 42, which can produce a multi-bit low data rate output to transmission circuitry 44. The data is remotely received by receiving circuitry 46. The received data may optionally be filtered further by digital filter 48, and this data may be processed by the FRI processing circuitry at 16 as described above. It will be appreciated that the transmit circuitry 44 and receive circuitry 46 can be placed at any point in the system of FIG. 4.

FIG. 5 illustrates another implementation of a circuit implementing the ΔΣ modulator 30. In this case, no low pass filtering of decimation is performed. Instead, a discrete Fourier transform (DFT) is performed at circuit 52 directly on the output bit stream from the ΔΣ modulator 30. The results of this DFT computation can provide the coefficients of the frequency components used by the FRI processing 16 to generate the FRI parameters. The transmission and reception of FIG. 4 can be implemented at any point in the system of FIG. 5 as in FIG. 4.

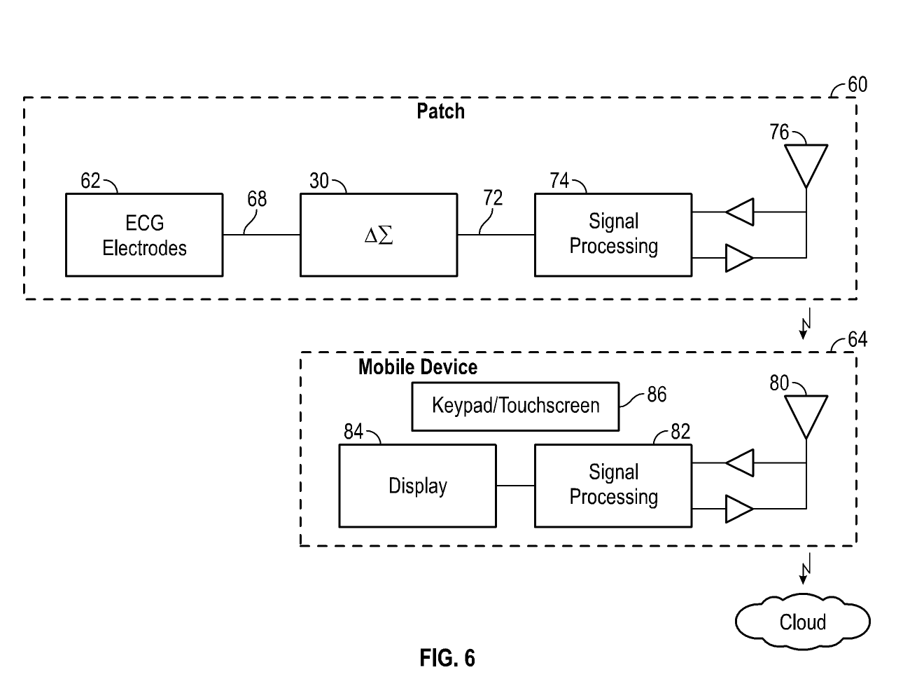

FIG. 6 illustrates a specific on-body monitoring apparatus in which the above described FRI methods can be implemented. Because biological signals and diagnostic measurement signals on biological systems may sometimes be characterized with FRI parameters, there has been interest in modeling such signals with FRI parameters for data acquisition, storage, and reconstruction purposes. There is further interest in using such techniques in ambulatory monitoring systems such as on-body ECG monitors, as implementation of FRI signal analysis can reduce the power and memory required to acquire, store, and transmit data representing measured biological signals and diagnostic information. As noted above, however, the sampling and acquisition of the digital data from the analog input signal without using a significant amount of power remains a problem in practical implementation of FRI processing in these devices.

In the system of FIG. 6, a patch ECG monitor 60 incorporates ECG electrodes 62 and is mounted with adhesive for example on a subject as an ambulatory cardiac monitoring device. Because the on-body mounted system 60 should use as little power as possible, it is advantageous to minimize the power consumption of the sampler and also minimize the amount of data that must be transmitted from the on-body system 60 to the mobile device 64. As described in U.S. patent application Ser. No. 13/552,206, filed on Jul. 18, 2012 and entitled System and Method for Analysis and Reconstruction of Variable Pulse-Width Signals with Finite Rates of Innovation, which is incorporated herein by reference in its entirety, ECG signals can be modeled using the principles of FRI signal analysis and processing as a sum of Lorentzian pulses, each having a position, width, and symmetric and asymmetric amplitude. This can be analyzed as a system with a low rate of innovation defined by these pulse parameters, but having a high bandwidth waveform, as the pulse widths in an ECG signal can be narrow. The Ser. No. 13/552,206 application describes a method of deriving these parameters from low frequency DFT coefficients of an input ECG signal. The sampling and FRI processing described herein and in the Ser. No. 13/552,206 application can reduce the power consumed by the on-body system 60 over many other signal acquisition and processing techniques.

In the system of FIG. 6, the ΔΣ modulator 30 operated as described above receives the ECG signal from line 68 without any significant amount of low pass filtering applied (although an input buffer/amplifier could be provided in line 68 which is not illustrated in FIG. 6). The output of the ΔΣ modulator 30 at line 72 is a 1 bit signal with content dependent on the signal input at line 68. This particular application of ΔΣ sampling and FRI processing may have characteristics similar to the hypothetical example presented above. As described in the Ser. No. 13/552,206 application, the FRI parameters for an ECG can be derived from frequency components in the original signal between 0 and 70 Hz or less, generally between 0 to anywhere from 50-70 Hz. The signal input to the ΔΣ modulator 30 (analog filtered or unfiltered from the ECG electrodes 62) may have 2-5 kHz maximum frequency, and the ΔΣ modulator 30 in FIG. 6 can be configured to sample the signal at a sampling rate of 4-10 kHz.

The samples output from the ΔΣ modulator 30 are sent to signal processing circuitry 74 which may be configured to produce the FRI parameters of, for example, pulse width, time, and symmetric and asymmetric amplitude in the manner described in the Ser. No. 13/552,206 application. These may be sent wirelessly via antenna 76 to a mobile device 64 such as a cell phone, tablet, or other portable electronic system, which receives the parameters via antenna 80 and routes the parameters to signal processing circuitry 82 in the mobile device 64. It will be appreciated that the components of the patch 60 need not be mounted together on the same physical substrate, but could be split up in a variety of ways.

The signal processing circuitry 82 in the mobile device 64 may be configured to reconstruct the ECG waveforms using FRI parameters. The reconstructed signal may be displayed on a display 84 and manipulated with a keypad/touchscreen 86 on the mobile device. The mobile device may also be configured to transmit either the reconstructed waveform and/or the FRI parameters to an external network such as the Internet for storage, review by a physician, etc.

The signal processing circuit 74 can process the output bit stream in a variety of ways. The output bit stream from the ΔΣ modulator 30 can be simply forwarded to the transmission circuitry, with all further processing performed on the mobile device 64. A DFT can be performed on the bit stream and the DFT coefficients for FRI processing can be forwarded for transmission. The DFT coefficients can be used to derive the appropriate FRI parameters as described above on the patch 60, and these parameters can be transmitted to the mobile device 64. In another implementation, the bit stream can be digitally low pass filtered in the time or frequency domain to produce multi-bit data points representing the signal level at discrete time points such as are obtained by a high resolution sampling analog to digital converter. These digitally filtered points can be further decimated without loss of information. A DFT can be performed on these data points and the FRI parameters can be produced for transmission to the mobile device 64.

The various illustrative logic, logical blocks, modules, and algorithm steps described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and steps described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. In some implementations, particular steps and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium. The steps of a method or algorithm disclosed herein may be implemented in a processor-executable software module which may reside on a computer-readable medium. Computer-readable media includes both computer storage media and communication media including any medium that can be enabled to transfer a computer program from one place to another. A storage media may be any available media that may be accessed by a computer. By way of example, and not limitation, such computer-readable media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection can be properly termed a computer-readable medium. Disk and disc, as used herein, includes compact disc (CD), laser disc, optical disc, digital versatile disc (DVD), floppy disk, and Blu-Ray™ disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. Additionally, other implementations are within the scope of the following claims. In some cases, the actions recited in the claims can be performed in a different order and still achieve desirable results.

What is claimed is:

1. A method for sparse sampling an analog signal comprising:
sampling and quantizing the analog signal with a delta-sigma modulator;
filtering the output of the delta-sigma modulator; and
deriving FRI parameters from the sampled, quantized, and filtered analog signal.

2. The method of claim 1, wherein the method includes determining DFT coefficients of the analog signal and using only DFT coefficients corresponding to a selected frequency band to derive the FRI parameters.

3. The method of claim 1, comprising performing a DFT on the output of the delta-sigma modulator.

4. The method of claim 1, comprising low pass filtering an input analog signal to produce the analog signal that is sampled with the delta-sigma modulator.

5. The method of claim 1, wherein the analog signal is an unfiltered analog signal from a signal source.

6. The method of claim 1, wherein the analog signal is a filtered or unfiltered ECG signal.

7. An apparatus for sparse sampling an analog signal comprising:
a delta-sigma modulator having an input coupled to receive the analog signal;
processing circuitry coupled to an output of the delta-sigma modulator, and configured to filter an output of the delta-sigma modulator and to derive FRI parameters from the output of the delta-sigma modulator.

8. The apparatus of claim 7, comprising a signal source generating the analog signal.

9. The apparatus of claim 8, wherein the signal source comprises a filter.

10. The apparatus of claim 7, comprising ECG electrodes.

11. The apparatus of claim 7, wherein at least some of the processing circuitry is located remotely from the delta-sigma modulator.

12. A method for sparse sampling an analog signal comprising:
sampling and quantizing the analog signal with a delta-sigma modulator;
determining a set of DFT coefficients from the output of the delta-sigma modulator; and
deriving FRI parameters from the set of DFT coefficients.

13. The method of claim 12, wherein the method includes determining DFT coefficients of the analog signal and using only DFT coefficients corresponding to a selected frequency band to derive the FRI parameters.

14. The method of claim 12, comprising low pass filtering an input analog signal to produce the analog signal that is sampled with the delta-sigma modulator.

15. The method of claim 12, wherein the analog signal is an unfiltered analog signal from a signal source.

16. The method of claim 12, wherein the analog signal is a filtered or unfiltered ECG signal.

17. An apparatus for sparse sampling an analog signal comprising:
a delta-sigma modulator having an input coupled to receive the analog signal;
processing circuitry coupled to an output of the delta-sigma modulator, and configured to determine a set of DFT coefficients from the output of the delta-sigma modulator and derive FRI parameters from the set of DFT coefficients.

18. The apparatus of claim 17, comprising a signal source generating the analog signal.

19. The apparatus of claim 18, wherein the signal source comprises a filter.

20. The apparatus of claim 17, comprising ECG electrodes.

21. The apparatus of claim 17, wherein at least some of the processing circuitry is located remotely from the delta-sigma modulator.
